(19) United States
(12) Patent Application Publication
(10) Pub. No.: US 2014/0243243 A1
(43) Pub. Date: Aug. 28, 2014

(54) DEVICE AND METHOD FOR CELL-EXCLUSION PATTERNING

(71) Applicant: Corning Incorporated, Corning, NY (US)

(72) Inventor: Elizabeth Tran, Painted Post, NY (US)

(73) Assignee: Corning Incorporated, Corning, NY (US)

(21) Appl. No.: 14/273,940

(22) Filed: May 9, 2014

Related U.S. Application Data
(63) Continuation of application No. 13/450,852, filed on Apr. 19, 2012.

(51) Int. Cl. C12M 1/32; G01N 33/50

(52) U.S. Cl. CPC C12M 23/12; G01N 33/5008
USPC 506/26; 435/305.2

(57) ABSTRACT

This disclosure relates to devices and methods for cell-exclusion patterning. Specifically, this disclosure provides a device and method to exclude cells in selected areas during cell seeding and create cell-free arrays that can be used for cell migration and related studies and assays.

### CROSS-REFERENCE TO RELATED APPLICATIONS

This application is a continuation-in-part of U.S. application Ser. No. 13/450,852, filed Apr. 19, 2012, the contents of which is relied upon and incorporated herein by reference in its entirety, and the benefit of priority under 35 U.S.C. §120 is hereby claimed.

### TECHNICAL FIELD

The present disclosure relates in general to a cell culture device that provides an area which contains cells and an area which is free of cells. In one application, the cell culture device can be used to perform assays to determine activity of a chemical entity, or cell motility assays.

### BACKGROUND

Cell migration and related processes are critical components of many physiologically important processes such as wound healing, angiogenesis, embryogenesis, cancer metastasis, and immune response. A variety of methods have been developed for studying the migratory behavior of cells. These methods fall into two categories: those involving devices that can generate chemical gradients, such as Transwell® (Corning, Incorporated, Corning, N.Y.) and Boyden chambers, and those involving devices that can create cell-free areas in cell monolayers.

The Transwell® and Boyden chambers, which involve the migration of cells through a microporous filter in response to a chemotactic gradient, are among the most commonly-used devices for studying cell migration and invasion. However, these devices are limited to the studies of cells that migrate individually or migrate in response to a chemical gradient, typically do not permit real-time viewing of the cells, and require the migrated cells be stained or lysed for analysis.

The creation of cell-free areas in cell monolayers is an important component of scratch/wound migration assays. The scratch/wound assays enable measurements of cell migration in the absence of a chemo-attractant. They often involve creating cell-free areas using tools such as a pipette tip, a syringe needle, a razor blade, electric current, and laser light. While enabling the monitoring of cellular responses in real-time, creating the cell-free area using these devices often results in damage to the cells, e.g., at the edge of the wound, and to the cell culture surface. In addition, the resulting cell-free areas are often inconsistent in one or more of size, shape and location.

More recently, several cell-exclusion patterning methods have been developed as improved alternatives to the scratch/wound methods. These tools include silicone stoppers, and biocompatible gel (BCG) to block the attachment of cells in predetermined areas during cell seeding. As with the scratch/wound methods, these patterning methods also involve the creation of cell-free areas in confluent cell monolayers. However, they do so using tools that minimize cell damage and create cell-free areas of uniform size and shape.

Both the silicone stoppers and BCG have been developed to provide a more reproducible alternative to the scratch/wound closure assay, and a less cumbersome method than Transwell®/Boyden chamber devices for cell migration studies. For assays where the cell culture surface is coated with an extracellular matrix (ECM), however, the direct contact of the silicone stoppers and BCG with the ECM, as required by these approaches, may result in alteration of the ECM structure. In addition, it may leave behind undesirable residues.

The following references describe the state of-the-art, the contents of which are hereby incorporated by reference in their entirety. WIPO International Publication No. WO2009/026359, US Patent Application Publication No. 2009/0054162, Rehydration of Polymeric, Aqueous, Biphasic System Facilitates High Throughput Cell Exclusion Patterning for Cell Migration Studies, Tavana et al., Adv. Funct. Mater. 2011, 21, 2920-2926, and A high-throughput cell migration assay using scratch wound healing, a comparison of image-based readout methods, Yarrow et al., BMC Biotechnology 2004, 4:21.

In view of the foregoing, it would be advantageous to provide improved approaches to cell-exclusion patterning.

### SUMMARY

The present disclosure relates to devices and methods for cell-exclusion patterning, and particularly devices and methods to exclude cells in selected areas during cell seeding so as to create cell-free arrays that can be used for cell migration and related studies and assays.

The disclosure provides a kit for cell-exclusion patterning that includes a multi-well culture plate comprising a plurality of wells. The multi-well plate includes a frame comprising at least a first side panel and a second side panel, wherein the first side panel optionally comprises a plurality of guide holes. The second side panel may also comprise a plurality of guide holes.

In embodiments, the kit further comprises a comb or a pin plate each comprising a plurality of blocking pins. The comb comprises a comb having a bar, at least one guide pin extending from the comb bar, and a plurality of blocking pins extending from the comb bar, each pin having a bottom surface. A comb comprises at most a single row of blocking pins. The at least one guide pin is structured and arranged to engage with a first side panel of the multi-well plate and the plurality of blocking pins are structured and arranged to extend into a plurality of respective wells. In an example embodiment, the comb comprises two guide pins, a first guide pin structured and arranged to engage with a first side panel of the multi-well plate, and a second guide pin structured and arranged to engage with a second side panel of the multi-well plate.

The plate comprises a plurality of blocking pins extending therefrom. A pin plate comprises plural rows of blocking pins. The pin plate is structured and arranged to engage with a first side panel of the multi-well plate such that the plural rows of blocking pins extend into a plurality of respective wells of the multi-well plate.

Thus, in one embodiment, disclosed is a kit for making a multi-well cell plate for cell-exclusion patterning. The kit includes (i) a comb comprising a comb bar, at least one guide pin extending from the comb bar, a plurality of blocking pins extending from the comb bar, each having a bottom surface; and (ii) a multi-well plate, wherein the multi-well plate comprises a plurality of wells, each well having side walls and a well bottom, a first side panel and a second side panel, wherein the first side panel comprises a plurality of guide holes.

The at least one guide pin is structured and arranged to engage with one of the plurality of guide holes of the first side panel of the multi-well plate such that when the at least one guide pin is engaged with one of the plurality of guide holes, the plurality of blocking pins are inserted into the plurality of wells.

In a further embodiment, the kit includes (i) a pin plate having plural rows of blocking pins extending from the pin plate, each blocking pin having a bottom surface; and (ii) a multi-well plate, wherein the multi-well plate comprises a plurality of wells, each well having side walls and a well bottom, a first side panel and a second side panel.

The pin plate is structured and arranged to engage with the multi-well plate in order to insert the blocking pins into respective ones of the plurality of wells of the multi-well plate. The pin plate may further comprise a plurality of access ports for seeding and feeding of cells into the wells.

The multi-well plate may comprise, for example, a 96-well plate or a 384-well plate. The blocking pins may be cylindrical (e.g., columnar) having any desired cross-section. The blocking pins may have a flat bottom surface. In embodiments, while engaged with the multi-well plate, the bottom surface of each blocking pins does not contact the respective well bottom, and thus provides a non-contact device for cell-exclusion patterning. In embodiments, the flat bottom surface of the blocking pins is parallel to the well bottom.

A method of using the kit for cell-exclusion patterning comprises (a) engaging the comb with the multi-well plate so that the at least one guide pin is engaged with one of the plurality of guide holes of the first side panel of the multi-well plate and the plurality of blocking pins are inserted into the plurality of wells, or engaging the pin plate with the multi-well plate so as to insert a plurality of blocking pins into a plurality of wells; (b) adding cell culture media to the wells of the multi-well plate; (c) adding cells to the wells of the multi-well plate; (d) allowing cells to settle and adhere to the bottom of the wells of the multi-well plate and (e) removing the comb or pin plate from the multi-well plate.

The comb enables patterning of a discrete number of wells within the multi-well plate, while the pin plate enables a higher throughput patterning of, for example, all or substantially all of the wells in the multi-well in a single step. A 96-pin plate or a 384-pin plate, for example, enables all of the pins to be handled as a single unit, and can be used to create cell-free areas without contacting the pins with the cell culture surface and/or without the need for subsequent cell washing.

Additional aspects of the disclosure will be set forth, in part, in the detailed description, figures and any claims which follow, and in part will be derived from the detailed description, or can be learned by practice of the various embodiments. It is to be understood that both the foregoing general description and the following detailed description are exemplary and explanatory only and are not restrictive of the embodiments as disclosed.

### BRIEF DESCRIPTION OF THE DRAWINGS

A more complete understanding of the disclosure may be had by reference to the following detailed description when taken in conjunction with the accompanying drawings wherein:

FIG. 1 is a perspective drawing illustrating a multi-well cell culture plate and a pin comb, which form an embodiment of the kit disclosed herein;

FIGS. 2A and 2B are illustrations of the comb engaged with the multi-well plate, where FIG. 2A is a cross-sectional view of a comb engaged with the multi-well plate and FIG. 2B is a blown-up illustration of the area 20 shown in FIG. 2A;

FIGS. 3A-E illustrate a method of using the multi-well plate and comb to create a cell-exclusion cell culture;

FIG. 4 is an exploded illustration of a kit comprising a lid, pin plate and multi-well plate, which form an embodiment of the kit disclosed herein;

FIGS. 5A and 5B illustrate example 96-well formatted pin plates according to embodiments;

FIG. 6 illustrates an example 384-well formatted pin plate;

FIG. 7 illustrates a comb configured to engage with a multi-well plate using guide holes provided in a frame;

FIG. 8 is a cross-sectional view of a pin plate engaged with a multi-well plate;

FIG. 9 is an illustration of the bottom of a cell culture well, having cells in a cell-exclusion ring;

FIGS. 10A-F are a series of micrographs showing cells growing in a cell-exclusion patterned cell culture in non-treated wells (NT) (A-C) and wells coated with Fibronectin (FN) (D-F);

FIGS. 11A-F are micrographs showing cells growing in a cell-exclusion patterned cell culture in the absence (A, C and E) and presence (B, D and F) of a pharmaceutical agent (in this case, CytoD) in an assay;

FIGS. 12A and 12B are micrographs showing cells growing in a cell-exclusion patterned cell culture in the presence (A) and absence (B) of a pin in the well;

FIGS. 13A-F are heat map representations of A549 cells cultured atop a Corning Epic® biosensor, illustrating that cells grow into the cell exclusion zone when cultured over time.

### DETAILED DESCRIPTION

This disclosure provides high-throughput cell-exclusion patterning devices and related methods to partition cells into cell-containing and cell-free areas during cell seeding so that subsequent migration and growth of cells from cell areas to cell-free areas can be observed, recorded and analyzed. In particular, this disclosure provides a device and method for patterning and depositing living cells in predetermined areas by a mechanism that does not involve direct contact of the device with the cell culture surface. The device and method therefore enable cell patterning without damage or change to either the cells or the cell culture surface. The device is simple and easy to use and comprises a set of equally spaced pins that 1) can be lowered into wells of microtiter plates so that the bottom surface of each pin is near but not touching the bottom of the wells, and 2) can function as a non-contact mask for blocking the deposition of cells in selected areas during cell seeding.

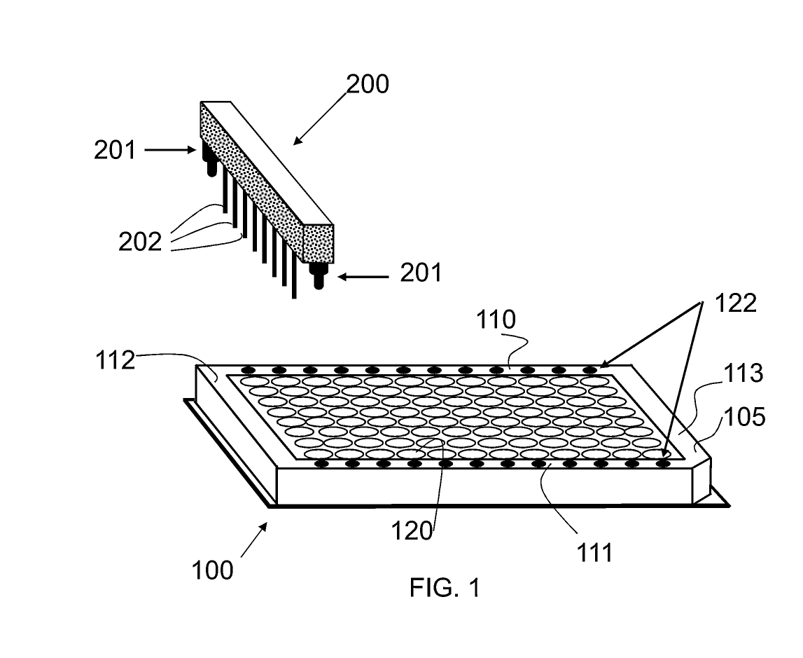

FIG. 1 is a perspective drawing illustrating a multi-well cell culture plate 100 and a comb 200, which form an embodiment of the cell-exclusion patterning kit disclosed herein. In embodiments, the multi-well plate 100 has a top surface. The top surface has at least a first panel 110 peripheral to a plurality of or an array of wells 120. In the embodiment shown, four panels 110, 111, 112, 113 are shown, forming a frame 105 surrounding the plurality or array of wells 120. In embodiments, at least one panel 110 comprises a plurality of guide holes 122. In embodiments, the multi-well plate comprises a plurality of wells 120, a frame 105 integral to the plate 100 comprising at least a first side panel 110, wherein the first side panel 110 comprises a plurality of guide holes 122. In embodiments, the frame comprises a second side panel 111 wherein the second side panel comprises guide holes 122.

FIG. 1 also illustrates an embodiment of a comb 200. The comb has at least one guide pin 201 and a plurality of blocking pins 202. The comb 200 is structured and arranged to engage with the multi-well plate 100 where the guide pin(s) 201 of the comb 200 engage with the guide hole(s) 122 of the multi-well plate 100. When the guide pins 201 of the comb 200 engage with the guide holes 122 of the multi-well plate 100, the blocking pins 202 insert into the plurality of wells 120 of the multi-well plate 100.

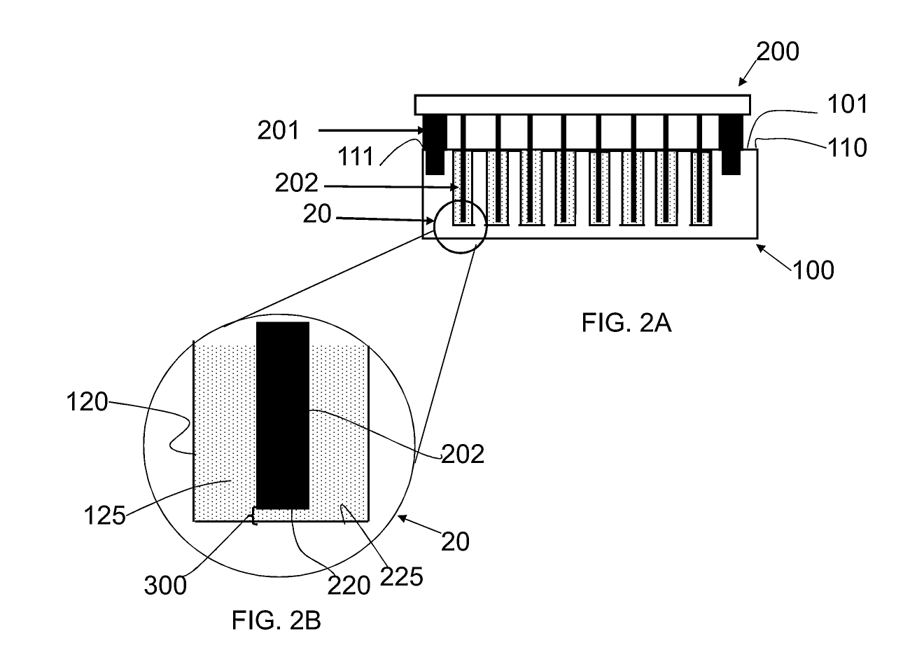

FIGS. 2A and 2B are illustrations of the comb 200 engaged with the multi-well plate 100. FIG. 2A is a cross-sectional view of a comb 200 engaged with the multi-well plate 100, and FIG. 2B is a blown-up illustration of the area 20 shown in FIG. 2A. As illustrated, guide pins 201 extend through the top surface 101 of the multi-well plate into first side panel 110 and second side panel 111. Guide pins 201 extend through the top surface into guide holes 122.

FIG. 2B illustrates blocking pin 202 extending into well 120. The well as illustrated is filled with media 125. When the comb 200 is engaged with the multi-well plate 100, and blocking pin 202 extends into the well 120, the blocking pin 202 does not engage with (i.e., does not physically touch) the bottom surface 225 of the well 120. That is, there is a gap 300 between the bottom surface 220 of the blocking pin 202 and the bottom surface 225 of the well 120. In embodiments, blocking pins 202 can be lowered into the wells of multi-well plate 100 so that the bottom surface 220 of each blocking pin 202 is near but not touching the bottom 225 of the well 120. A gap length can range from about 10 microns to 200 microns, e.g., 10, 20, 50, 100 or 200 microns. In embodiments the gap length is less than 100 microns.

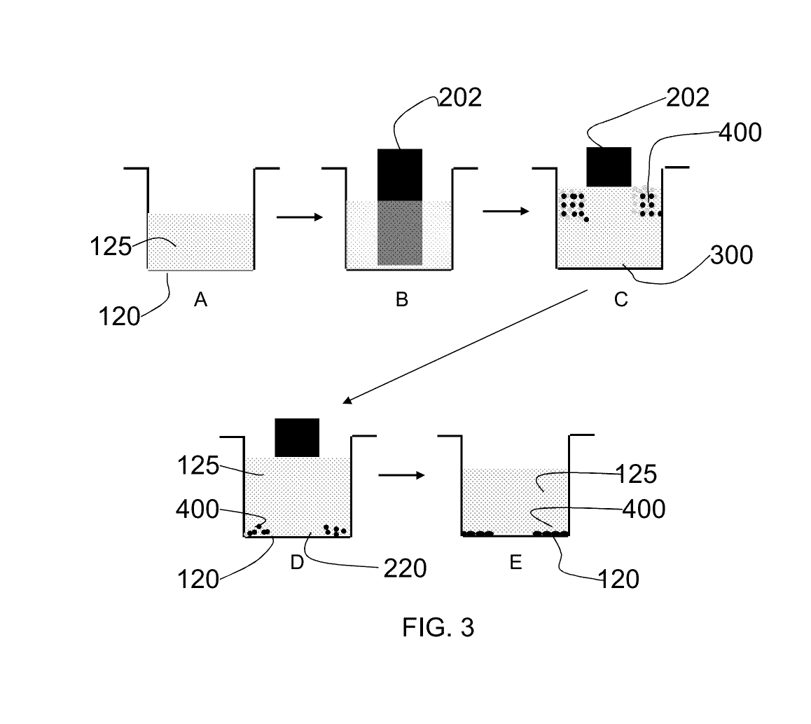

FIGS. 3A-E illustrate a method of using the multi-well plate 100 and comb 200 to create a cell-exclusion cell culture. In Step A, culture media 125 is added to the wells 120 of a multi-well plate. Media 125 is added to the wells 120 before blocking pins 202 are introduced into the wells 120 so that air bubbles do not form between the blocking pins 202 and the well bottom 225. Once the wells contain culture media 125, blocking pins are lowered into the wells 120, e.g., into the center of each respective well (Step B). Note that the blocking pin 202 is inserted so as to leave a gap 300 between the bottom surface of the well 225 and the bottom surface 220 of the blocking pin 202. In Step C, cells 400 can be added via a cell dispersion using, for example, a multi-channel pipettor or an automated liquid dispensing system. After the cells 400 settle and adhere to the bottom 225 of the wells 120, in Step D, the comb 200 is removed (Step E) to provide well-defined cell-containing and cell-free areas that can be used for cell migration and related studies.

The blocking pins 202 serve to block the deposition of cells during cell seeding, and hence create cell-free areas whose shape and size depend on the shape and size of the pins. In embodiments, the blocking pins can be of any cross-sectional shape, for example, round, oval, triangular, square, rectangular, and the like.

The guide pins 201 center the blocking pins in each corresponding well, fix the height of the blocking pins, prevent them from touching the bottom of the wells, and prevent the insert and hence the blocking pins from becoming dislodged during cell seeding and other handling steps.

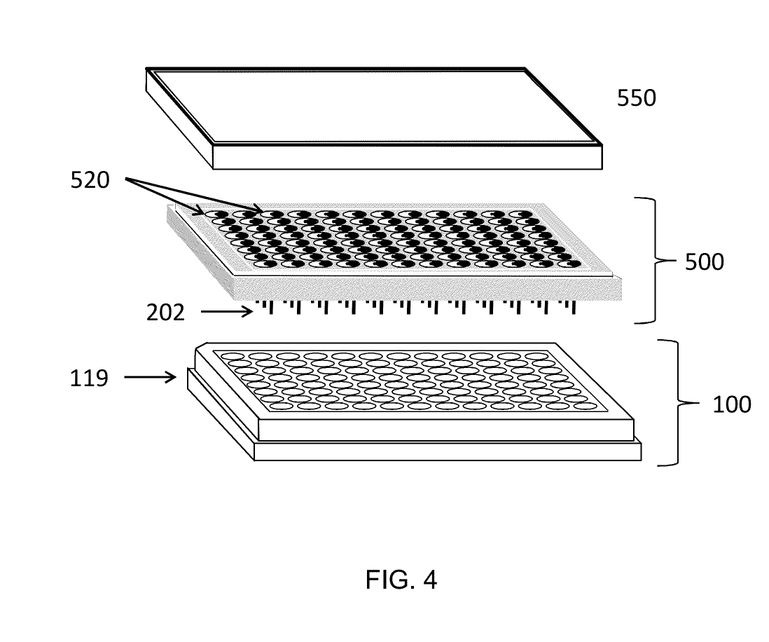

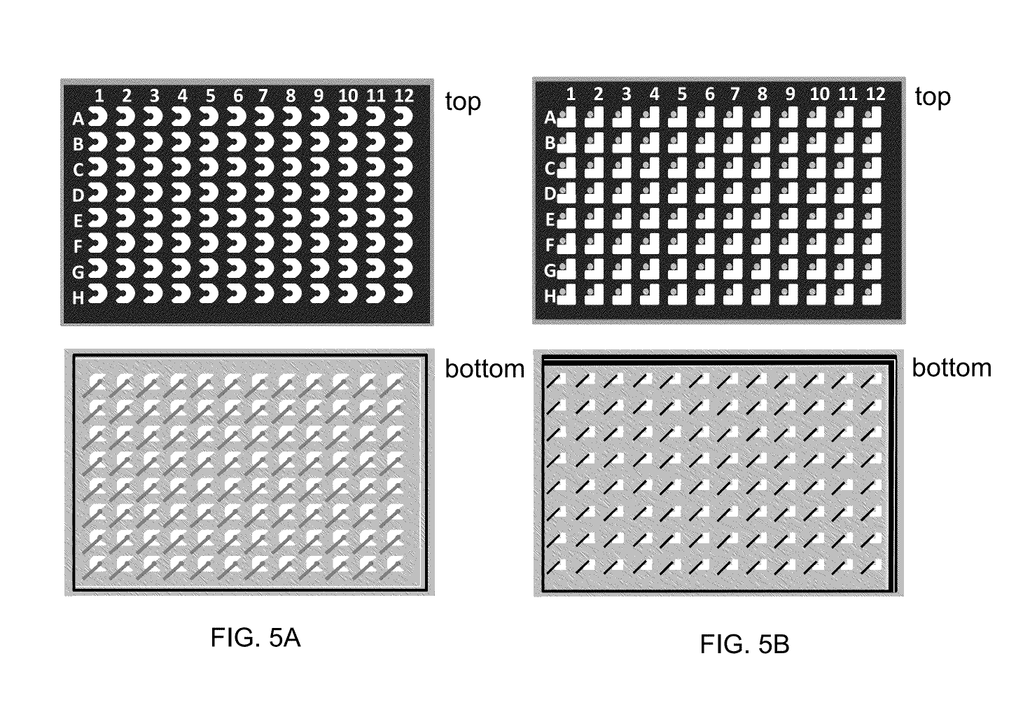

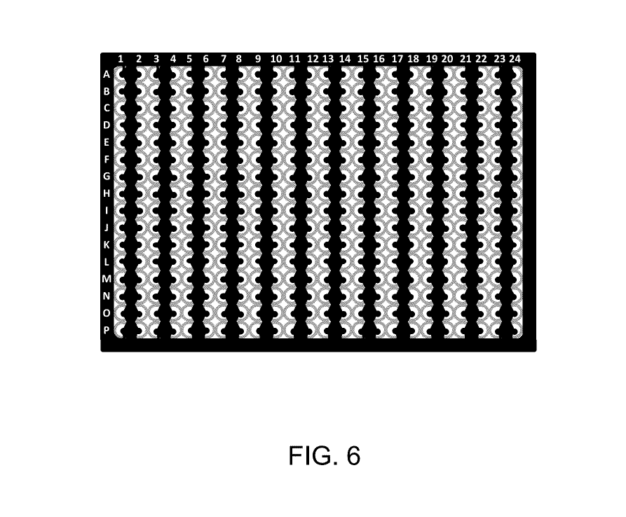

FIG. 4 is a perspective drawing illustrating a multi-well cell culture plate 100 and a pin plate 500 according to a further embodiment. The illustrated multi-well plate is a 96-well skirted plate formatted according to the standards of the Society of Biomolecular Screening (SBS). Alternative microtiter plates may have 6, 24, 384 or even 1536 wells, e.g., arranged in a 2:3 rectangular array. Example 96-well formats are shown in FIGS. 5A and 5B, illustrating respective top and bottom views. In FIG. 5A, for example, the pin plate and the blocking pins may be formed from the same material as a single unit. In FIG. 5B, the blocking pins may be formed separately and attached to the pin plate. An example 384-well format is illustrated in FIG. 6.

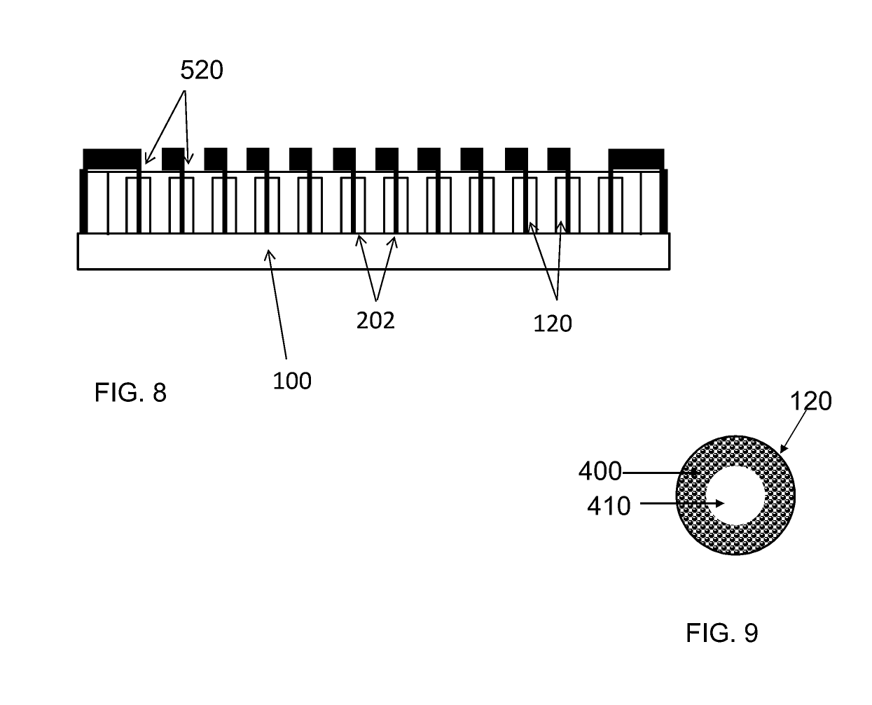

Referring to FIG. 4, pin plate 500 includes a plurality of blocking pins 202, each with an adjacent access port 520 for cell addition and feeding. Also illustrated is a lid 550 that conforms to the dimensions of the multi-well plate 100 and the pin plate 500. The lid is removable and includes a frame with a rigid planar surface and side walls extending from the periphery of the planar surface. The lid can be used to cover the pin plate and mitigate evaporation and contamination during assays. A cross-sectional schematic of the pin plate 500 showing the blocking pins 202 lowered into respective wells in the culture plate 100 is shown in FIG. 8. The blocking pins 202 are designed to align with the well centers, while the adjacent access ports 520 are designed to be in the same quadrant for all wells across the well plate.

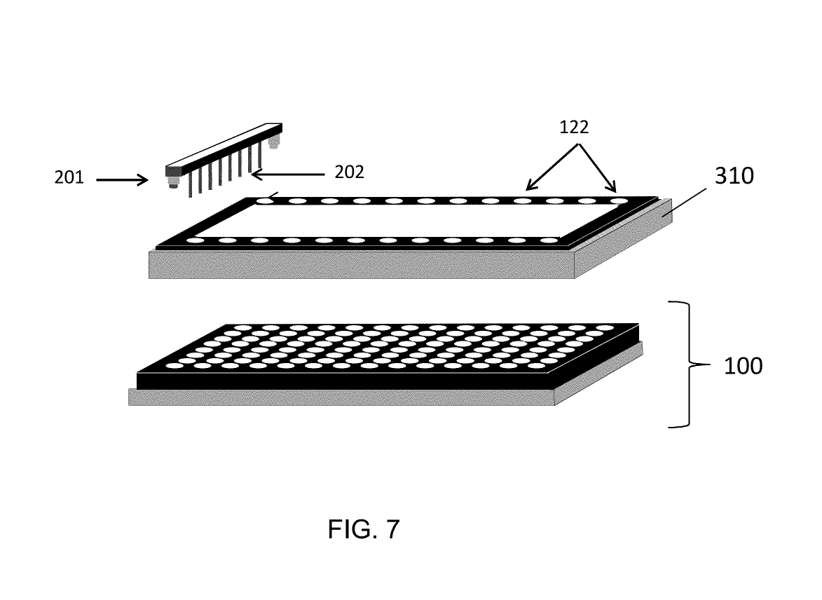

A kit embodiment including an alignment frame is illustrated in FIG. 7. In lieu of guide holes 122 formed in the culture plate, as illustrated in FIG. 1, an alignment frame 310 comprising a plurality of guide holes 122 can be used to position a comb (or pin plate, not illustrated) including a respective plurality of blocking pins 202 with respect to a culture plate 100 including a respective plurality of wells. The alignment frame 310, as illustrated, is configured to be arranged peripheral to the culture plate 100. With the alignment frame 310 engaged with the culture plate 100, blocking pins 202 can be lowered into the wells of multi-well plate 100.

The pin plate is a rigid structure that can be lowered into or removed from the well plate. By holding the skirt of the well plate and the sidewalls of the pin plate during these processes, the two plates can be joined or separated from each other. The pin plate 500 has sidewalls with dimensions that serve several important functions: 1) they allow the pin plate to sit form-fittingly on the skirt of the well plate and prevent the blocking pins from becoming dislodged during cell seeding and other handling, 2) they allow the blocking pins and adjacent access ports to align with the corresponding wells, and 3) they allow the tip of each blocking pin to be positioned proximate, but not in contact with the well bottom. The pin plate's sidewalls thus act as the guiding tabs for the blocking pins and access ports. A method of using the multi-well plate 100 and pin plate 500 is essentially as described above with reference to FIG. 3 for the comb. A 96-pin plate or a 384-pin plate, for example, enables all of the pins to be handled as a single unit and, in contrast to a comb, can be used to seed an entire multi-well plate in a single step, i.e., without the need to relocate the blocking pins 202.

Compared to existing technologies for cell-exclusion patterning, which rely on the direct contact of seeding stoppers and biocompatible gel (BCG) deposits with a well-bottom, the blocking pin insertion devices described here offer a non-contact method that can exclude cells in selected areas during cell seeding. The comb and pin plate thus have all of the advantages of the seeding stoppers and BCG deposits. For example, like seeding stoppers and BCG deposits, the comb and plate yield cell-free areas with consistent shape, size and location, avoid cell damage, are simple and easy to use, compatible with adherent cells, and are suitable for subsequent high-throughput screening (HTS) and high-content analysis (HCA).

In contrast to seeding stoppers and BCG deposits, the comb and plate (because they are non-contact) do not interfere with the cell culture surface, and are suitable for patterning non-adherent cells. Further, both the comb and plate allow subsequent cell migration studies to be carried out with or without the blocking pins still present in the wells. For instance, if the wells are coated with a material to enhance cell culture, the insertion of a blocking pin that comes into contact with the cell bottom might disrupt the coating. In addition, the addition of a BCG may result in an undesired BCG residue.

Using the comb or pin plate and multi-well devices embodied herein, it is possible to form a cell exclusion cell culture, while preserving a cell culture surface on the bottom of a well. As shown in FIG. 3, when the blocking pins are hollow, the cell-free areas can still be viewed by microscope even when the blocking pins are inside the wells.

FIG. 9 is an illustration of the bottom of a cell culture well 120 formed using the method described in FIG. 3, having cells 400 in a cell-inclusion ring, surrounding a cell-exclusion area 410. The illustration of FIG. 9 may be formed using either a comb or a pin plate.

In embodiments, the multi-well plate, lid and the comb or pin plate (including blocking pins) can be formed from any suitable material including plastic, glass, glass-ceramic, metal, metal alloy, or combinations thereof. Suitable plastic materials include polystyrene, polycarbonate, acrylic, polystyrene, or polyester, or any other polymer suitable for molding and commonly utilized in the manufacture of laboratory ware.

The blocking pins may be solid or hollow. The comb and pin plate may be disposable or autoclavable. A disposable comb or pin plate may be formed from material that is less durable than a reusable, autoclavable material. For example, a reusable, autoclavable comb or pin plate may be formed from metal, while a disposable comb or pin plate may be formed from plastic material. In embodiments, one or more of the multi-well plate, lid, comb or pin plate are optically transparent.

The comb (or pin plate) and blocking pins may comprise a unitary part, such as an injection molded part, where the comb (or pin plate) and the blocking pins are formed from the same material as a single unit. Alternatively, blocking pins may be formed separately and attached to a comb or pin plate. In such a case, where the blocking pins are formed separately and attached to a comb or pin plate, the blocking pins may formed from the same material as the comb or pin plate or from different materials. For example, a comb or pin plate may be formed from an injection molded plastic and the blocking pins may be formed from a ceramic, glass-ceramic, polymer, metal or metal alloy such as tungsten carbide or stainless steel.

The comb, with guide pins that can fit snuggly into guide holes in the frame of a multi-well plate serves several functions including: (1) centering the blocking pins in each corresponding well; (2) fixing the height of the blocking pins and preventing them from touching the bottom of the wells; and (3) preventing the blocking pins from becoming dislodged during cell seeding and other handling steps.

Embodiments described herein will be further clarified by the following examples.

### EXAMPLES

### Example 1

### Cell Culture

Materials. Cytochalasin D was obtained from Tocris Bioscience and dissolved in dimethylsulfoxide to give 50 mM stock solutions.

Human lung carcinoma cell line A549 and human cervical carcinoma cell line HeLa were obtained from American Type Culture Collection (ATCC). Both cell lines were cultured in Dulbecco's Modified Eagle Medium (DMEM) supplemented with 10% fetal bovine serum and 1% penicillin-streptomycin (complete medium).

### Example 2

### Cell-Exclusion Patterning

Cell-exclusion patterning was carried out by a sequence of steps, as illustrated in FIG. 3. In Step A, complete medium was added to wells of a microtiter plate. The volume added was approximately 30 ul for 384-well plates or 75 ul for 96-well plates. In step B, a comb having guide pins and blocking pins was lowered into the microtiter plate and the blocking pins were locked into the desired wells and the guide pins into the corresponding guide holes. In step C, cells were introduced into the wells of the microtiter plate by adding a solution containing cells to the walls of the wells. The volume of the cell solution was approximately 20 ul for 384-well plates or 25-50 ul for 96-well plates. The cell density was 8000 cells per well and 30,000 cells per well for 384- and 96-well plates, respectively. In step D, the cells were allowed to settle and adhere to the well bottom inside a cell culture hood for about 45 min. The microplates were then placed inside a humidified incubator at 37° C. and 5% CO₂ for 3-4 h to allow the cells to attach to the well bottom. In step E, the comb and the pins were removed from the wells. Drugs were then added, if necessary to carry out an assay.

### Example 3

### Imaging Cell Patterns

Cell patterns were imaged by bright-field or fluorescent microscopy or by using Corning Epic® label-free high-resolution optical resonance detection system (available from Corning Incorporated, Corning, N.Y.). The Epic® detection platform includes an optical detection unit and a 384-well microplate with resonant waveguide grating biosensors embedded in the bottom of each well. The optical detection unit measures changes in the local index of refraction due to the presence of cells and changes in cell response at the sensor surface.

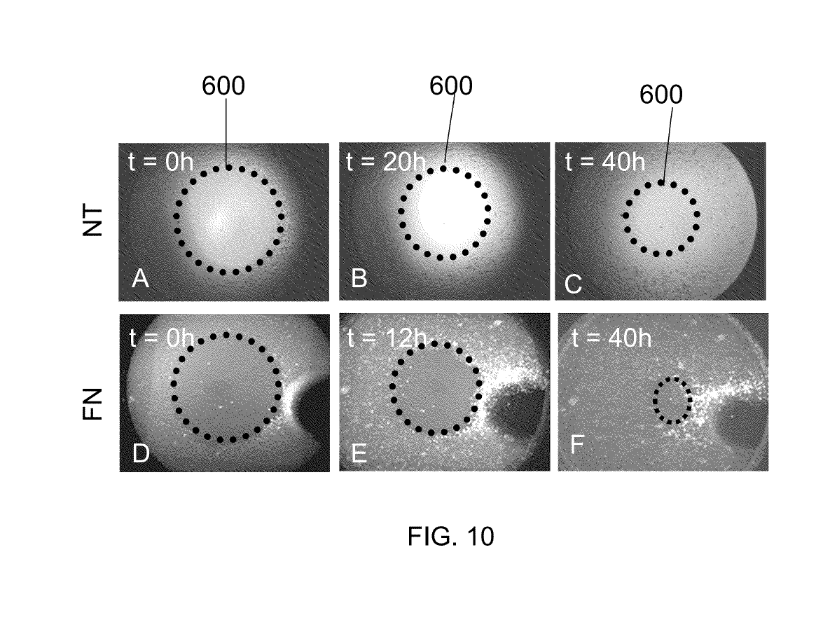

FIGS. 10A-G are a series of micrographs showing cells growing in a cell-exclusion patterned cell culture in non-treated microtiter wells (NT) (A-C) and in microtiter wells coated with Fibronectin (FN) (E-G) at time 0 (t=0 h) (A and D), time 12 hours (t=12 h) (E), time 20 hours (t=20 h) (B), and time 40 hours (t=40 h) (C and F). FIG. 10 shows that, over time, cells grow into the cell exclusion area 600 shown by the circle in FIGS. 10A, B, D, E and F. The structure on the right lower center side of FIGS. 10D, E, and F are bubbles. These are considered artifacts.

### Example 4

### Cell Culture in Cell-Exclusion Patterned Cell Culture in the Presence and Absence of a Drug

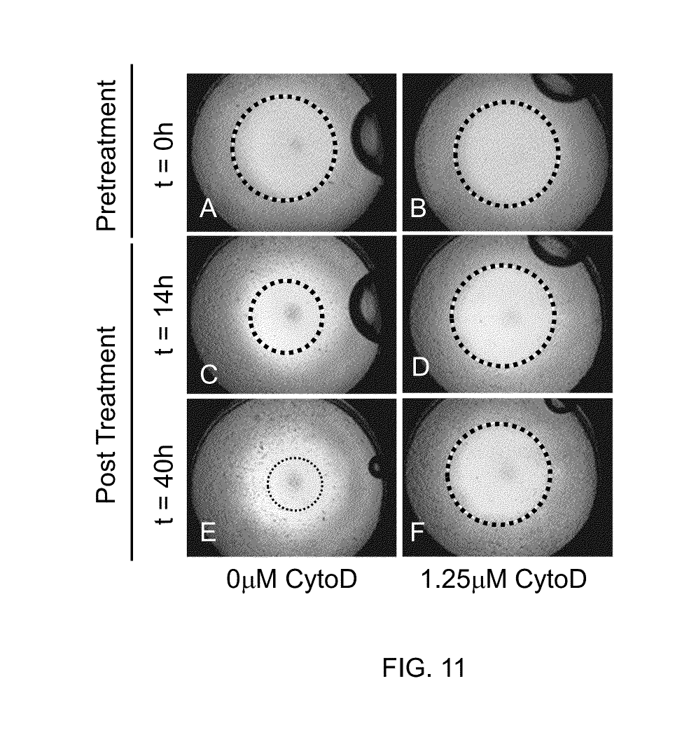

Cytochalasin D (CytoD) was added to wells to assess the effect of this drug on cell proliferation and migration. FIGS. 11A-F are micrographs showing cells growing in a cell-exclusion patterned cell culture on uncoated well bottoms in the absence (A, C, and E) and presence (B, D and F) of a pharmaceutical agent (in this case, CytoD) in an assay. Images were taken pretreatment (at t=0 h) (A and B), after 14 hours of treatment (t=14 h) (C and D), and after 40 hours of treatment (t=40 h) (E and F). FIG. 11 illustrates that, in the presence of CytoD, cells do not grow into the cell exclusion area over time.

### Example 5

### Cell Culture in the Presence and Absence of a Blocking Pin

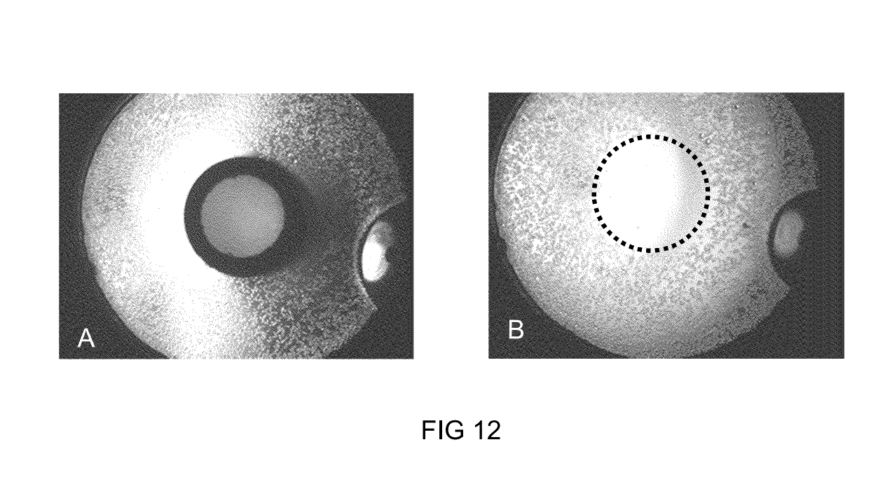

FIGS. 12A and 12B are micrographs showing cells growing in a cell-exclusion patterned cell culture in the presence (A) and absence (B) of a pin in the well. In the embodiment shown in FIG. 12A, the blocking pin diameter is 0.8 mm. The embodiment shown in FIG. 12B was formed using a blocking pin having a diameter of 0.8 mm. The structure on the right side is a bubble, and is considered to be an artifact of the image.

### Example 6

### Resonant Wavelength Distribution Heat Maps Characterizing A549 Cells Cultured Atop a Corning Epic® Biosensor

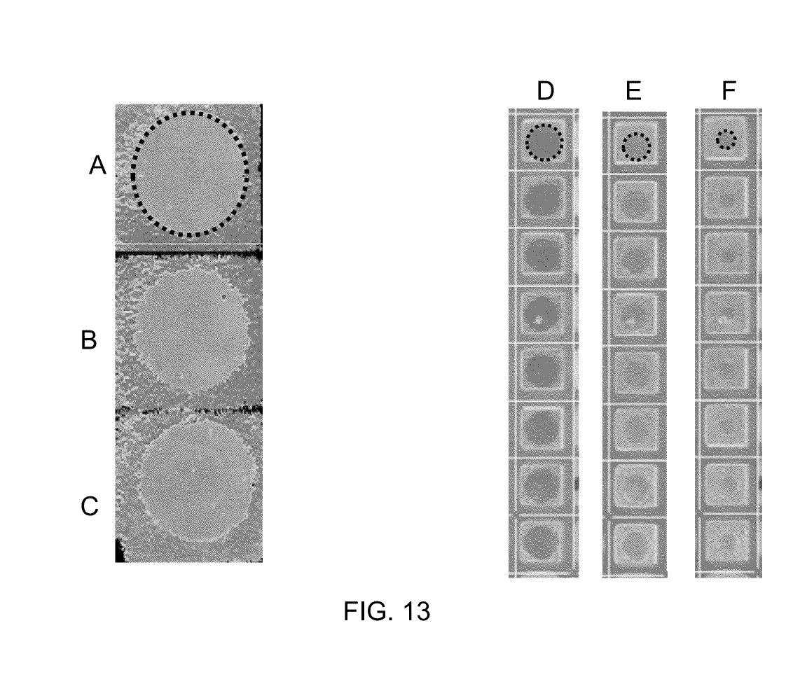

A549 cells were seeded at 8000 cells/well. Resonant wavelength distribution heat maps reflecting the growth and migration of A549 on an Epic® biosensor were captured using Corning EPIC® label-free high-resolution optical resonance detection systems with optical resolutions of 12 microns (FIGS. 13A, B and C) and 90 microns (FIGS. 13D, E and F), respectively.

FIGS. 13D-F are a series of resonant wavelength distribution heat maps showing A549 cells growing in a cell-exclusion patterned cell culture inside an Epic® microplate at time 0 (t=0 h) (D), time 24 hours (E), and time 48 hours (F).

Disclosed is a high-throughput device and method to partition cells into cell-containing and cell-free areas during cell seeding so that subsequent cell migration can be observed, recorded, and analyzed. In particular, the disclosure provides a simple and easy-to-use pin comb or pin plate for patterning cells in multi-well plates without damage or change to either the cells or the cell culture surface. The pin plate, for example, is a lid-like device that conforms to multi-well plates and that comprises a like number of pins for blocking the deposition of cells into the well centers. A pin plate may further comprise plural access ports for seeding and feeding of cells into the wells. The device is suitable for real-time and endpoint measurements, and is compatible with plate readers and image-based detection systems. The device may be used with co-cultures.

As used herein, the singular forms “a,” “an” and “the” include plural referents unless the context clearly dictates otherwise. Thus, for example, reference to a “blocking pin” includes examples having two or more such “blocking pins” unless the context clearly indicates otherwise.

Ranges can be expressed herein as from “about” one particular value, and/or to “about” another particular value. When such a range is expressed, examples include from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that any particular order be inferred. Any recited single or multiple feature or aspect in any one claim can be combined or permuted with any other recited feature or aspect in any other claim or claims.

It is also noted that recitations herein refer to a component being “configured” or “adapted to” function in a particular way. In this respect, such a component is “configured” or “adapted to” embody a particular property, or function in a particular manner, where such recitations are structural recitations as opposed to recitations of intended use. More specifically, the references herein to the manner in which a component is “configured” or “adapted to” denotes an existing physical condition of the component and, as such, is to be taken as a definite recitation of the structural characteristics of the component.

While various features, elements or steps of particular embodiments may be disclosed using the transitional phrase “comprising,” it is to be understood that alternative embodiments, including those that may be described using the transitional phrases “consisting” or “consisting essentially of,” are implied. Thus, for example, implied alternative embodiments to a well comprising culture media and cells include embodiments where a well consists of culture media and cells and embodiments where a well consists essentially of culture media and cells.

It will be apparent to those skilled in the art that various modifications and variations can be made to the present invention without departing from the spirit and scope of the invention. Since modifications, combinations, sub-combinations and variations of the disclosed embodiments incorporating the spirit and substance of the invention may occur to persons skilled in the art, the invention should be construed to include everything within the scope of the appended claims and their equivalents.

1. A kit for cell-exclusion patterning comprising:
a multi-well plate comprising a plurality of wells, a first side panel and a second side panel; and
a pin plate comprising a plurality of blocking pins extending therefrom,
wherein the plurality of blocking pins are structured and arranged to extend into a plurality of wells of the multi-well plate.

2. The kit of claim 1, wherein the multi-well plate is a 96-well plate or a 384-well plate.

3. The kit of claim 1, wherein the pin plate comprises a plurality of access ports structured and arranged to add cell culture media or cells into the wells of the multi-well plate.

4. The kit of claim 1, wherein the blocking pins are cylindrical.

5. The kit of claim 1, wherein each blocking pin has a flat bottom surface.

6. The kit of claim 1, wherein the blocking pins are solid.

7. The kit of claim 1, wherein the blocking pins are hollow.

8. The kit of claim 1, wherein the pin plate comprises a 96-pin plate or a 384-pin plate.

9. The kit of claim 1, where the pin plate and blocking pins are formed as a single unit from the same material.

10. The kit of claim 1, where the pin plate and blocking pins are formed as separate parts from the same material.

11. The kit of claim 1, where the pin plate and blocking pins are formed as separate parts from different materials.

12. The kit of claim 1, wherein the blocking pins are formed from a ceramic, glass-ceramic, polymer, metal or metal alloy.

13. The kit of claim 1, further comprising a lid configured to cover the pin plate and mitigate at least one of evaporation and contamination of culture media in the wells.

14. A method of using the kit of claim 1 for making a multi-well cell culture plate for cell-exclusion patterning comprising:
engaging the pin plate with the multi-well plate so that the plurality of blocking pins are inserted into respective ones of the plurality of wells;
adding cell culture media to the wells of the multi-well plate;
adding cells to the wells of the multi-well plate;
allowing cells to settle and adhere to the bottom of the wells of the multi-well plate; and
removing the pin plate from the multi-well plate.

15. The method of claim 14, wherein the blocking pins do not physically contact a surface of the multi-well plate.
